(19) United States
(12) Patent Application Publication
(10) Pub. No.: US 2018/0318135 A1
(43) Pub. Date: Nov. 8, 2018

(54) NOTIFICATION SERVICE PROVISION METHOD FOR HEARING-IMPAIRED PERSON AND DEVICE FOR EXECUTING SAME

(71) Applicant: UFIRST INC., Ansan-si (KR)

(72) Inventor: Hyun Sang LEE, Seongnam-si (KR)

(21) Appl. No.: 15/771,087

(22) PCT Filed: Mar. 8, 2016
(86) PCT No.: PCT/KR2016/002303

§ 371 Date: Apr. 25, 2018

(30) Foreign Application Priority Data
Oct. 26, 2015 (KR) 10-2015-0148985
Mar. 7, 2016 (KR) 10-2016-0026831

(51) Int. Cl. A61F 11/04; H04R 1/10; H04R 5/033; H04R 25/00

(52) U.S. Cl. CPC A61F 11/045; H04R 1/1075; H04R 2460/13; H04R 25/00; H04R 5/0335

(57) ABSTRACT

A notification service provision method for a hearing-impaired person executed on a notification service provision device for a hearing-impaired person, according to one embodiment of the present invention, comprises the steps of: checking whether the sensitivity of a sound received from a surrounding area through each of sound detection modules is equivalent to or greater than a preset vibration generation reference sensitivity; if, as a result of the checking, the sensitivity of the sound received through each of the sound detection modules is equivalent to or greater than the preset vibration generation reference sensitivity, calculating the sensitivity gap of the sound received through each of the sound detection modules; and according to the sound sensitivity gap, notifying a hearing-impaired person of the fact that a sound has occurred by operating at least one of vibration generation modules. Thus, the present invention has a merit of providing help to a hearing-impaired person by notifying the hearing-impaired person about a direction by means of vibration if the sensitivity of a sound collected from a surrounding area is greater than a sound sensitivity preset by the hearing-impaired person.

### CROSS-REFERENCE TO RELATED APPLICATIONS

This patent application is a national-stage application of International Patent Application No. PCT/KR2016/002303 which claims priority under 35 U.S.C. § 119 to Korean Patent Application No. 10-2015-0148985, filed on Oct. 26, 2015 and No. 10-2016-0026831, filed on Mar. 7, 2016 in the Korean Intellectual Property Office, the inventions of which are incorporated by reference herein in their entireties.

### TECHNICAL FIELD

Embodiments of the present invention relate to notification service providing methods for the hearing-impaired and devices for executing notification services.

### DISCUSSION OF RELATED ART

Hearing impairment refers to a partial or total inability to hear, and persons suffering from hearing impairment are called the hearing impaired.

With the development of state-of-the-art information communication equipment, there are ongoing research efforts for assistant equipment for deaf persons. Recently, automated sign language transmission systems, real-time text translation programs, and hearing aids are also being developed.

Automated sign language transmission systems are unable to precisely depict hand or finger gestures or facial expressions if sign language videos are transmitted on a low-frequency bandwidth through a regular transmission algorithm and thus cannot convey meaning for mutual communication. Although the video compression issue is addressed, the power issue—i.e., the system will consume significant more power for video compression-still remains.

Real-time text translation programs take such approach as translators type and translate, in real-time, speeches from, e.g., conferences, performances, lectures, or other various events, and transfer the translations to deaf persons' mobile phones. Such approach requires labor and thus cannot individually serve the deaf.

Hearing aids, by their structure, are uncomfortable to wear and have limitations to adjusting volume fitting various environments and extracting voice from sound.

Korean Patent Application Publication No. 10-2010-0030959 discloses a communication system for the hearing impaired, which consists of a remote translation center where a sign language translator resides and a portable communication device allowing the deaf person to remotely access the translation center to perform sign language communication via video and which enables smooth and precise communication with the user located close or far away. However, the patent nowhere discloses a scheme for addressing the above-mentioned problems.

### SUMMARY

The present invention aims to provide a notification service providing method for hearing-impaired persons that may indicate the direction when the sensitivity of sound gathered exceeds a set value and a device for executing the same.

The present invention also aims to provide a notification service providing method for hearing-impaired persons that allows an earphone to function as a hearing aid when the sensitivity of ambient sound gathered exceeds a set value, thereby freeing them from the inconvenience of wearing the hearing aid all the time and a device for executing the same.

Objectives of the present invention are not limited to the above objects, and other unmentioned objects are apparent to one of ordinary skill in the art from the following detailed description.

According to embodiments, a method for providing a notification service for hearing-impaired persons, executed on a sound alerting apparatus comprises determining whether sensitivities of ambient sounds respectively received through sound sensors are a preset vibration reference sensitivity or higher, calculating a gap between the sensitivities when the sensitivities are the preset vibration reference sensitivity or higher, and operate at least one of vibration generators according to the gap to alert a user to a sound occurrence.

According to embodiments, a sound alerting apparatus comprises microphones receiving and processing ambient sounds, vibration generators each operated under a control signal to generate a vibration, and a controller determining whether sensitivities of the ambient sounds respectively received from the microphones are a preset vibration reference sensitivity or higher, calculating a gap between the sensitivities when the sensitivities are the preset vibration reference sensitivity or higher, and operating at least one of the vibration generators according to the gap to alert a user to a sound occurrence.

The present invention may indicate the direction when the sensitivity of sound gathered exceeds a set value and a device for executing the same.

The present invention allows an earphone to function as a hearing aid when the sensitivity of ambient sound gathered exceeds a set value, thereby freeing them from the inconvenience of wearing the hearing aid all the time and a device for executing the same.

### BRIEF DESCRIPTION OF THE DRAWINGS

FIG. 1 is a view illustrating a network configuration for a system for providing a notification according to an embodiment of the present invention;

FIG. 2 is a block diagram illustrating a sound alerting apparatus according to an embodiment of the present invention;

FIGS. 3 to 5 are views illustrating a sound alerting apparatus implemented as a neckband according to an embodiment of the present invention;

FIG. 6 is a view illustrating a sound alerting apparatus implemented as a clip according to an embodiment of the present invention;

FIGS. 7 and 8 are views illustrating screenshots of an application running on a terminal according to an embodiment of the present invention.

### DETAILED DESCRIPTION OF EXEMPLARY EMBODIMENTS

Hereinafter, embodiments of the present invention are described in detail with reference to the accompanying drawings.

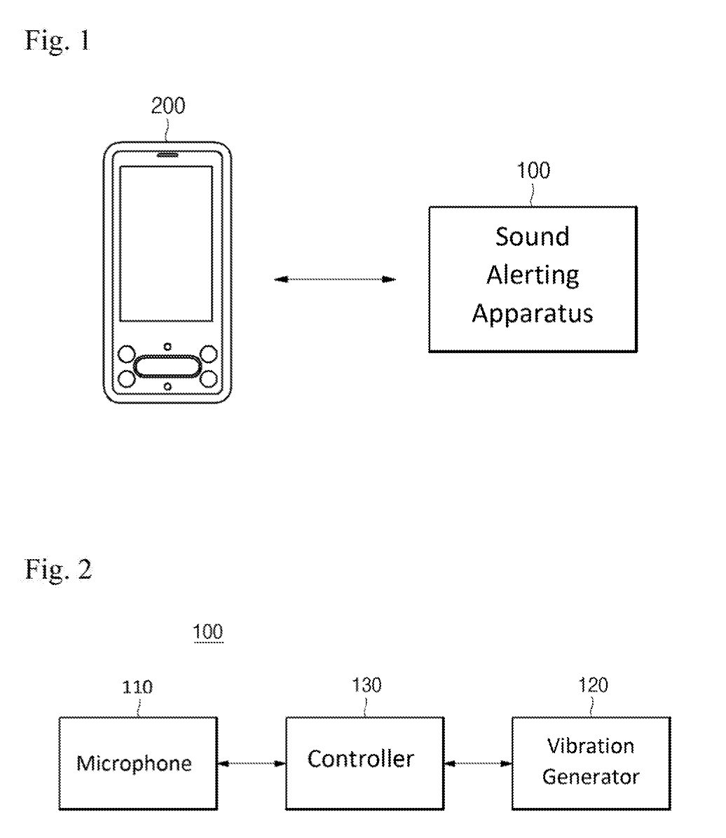

FIG. 1 is a view illustrating a network configuration for a system for providing a notification according to an embodiment of the present invention.

The system for providing a notification (hereinafter, simply referred to as a “system”) or the sound alerting apparatus according to the present invention may be more readily and usefully intended for hearing-impaired persons, but embodiments of the present invention are not essentially limited thereto.

Referring to FIG. 1, the system includes a sound alerting apparatus 100 and a terminal 200.

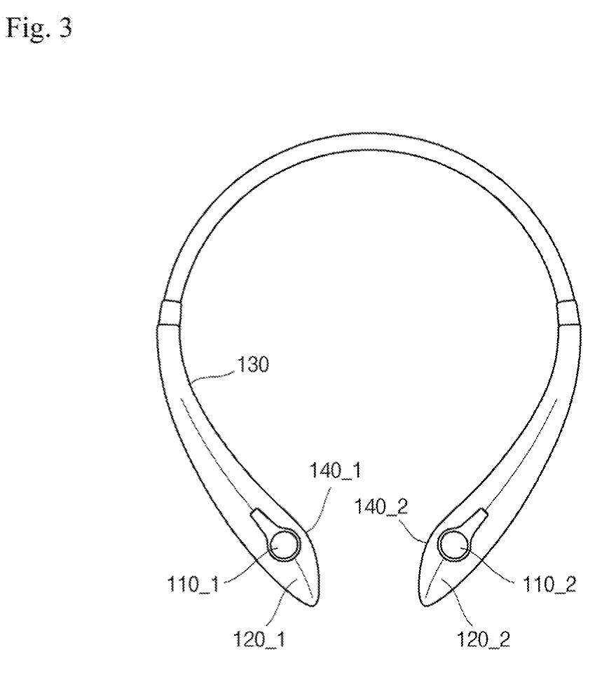

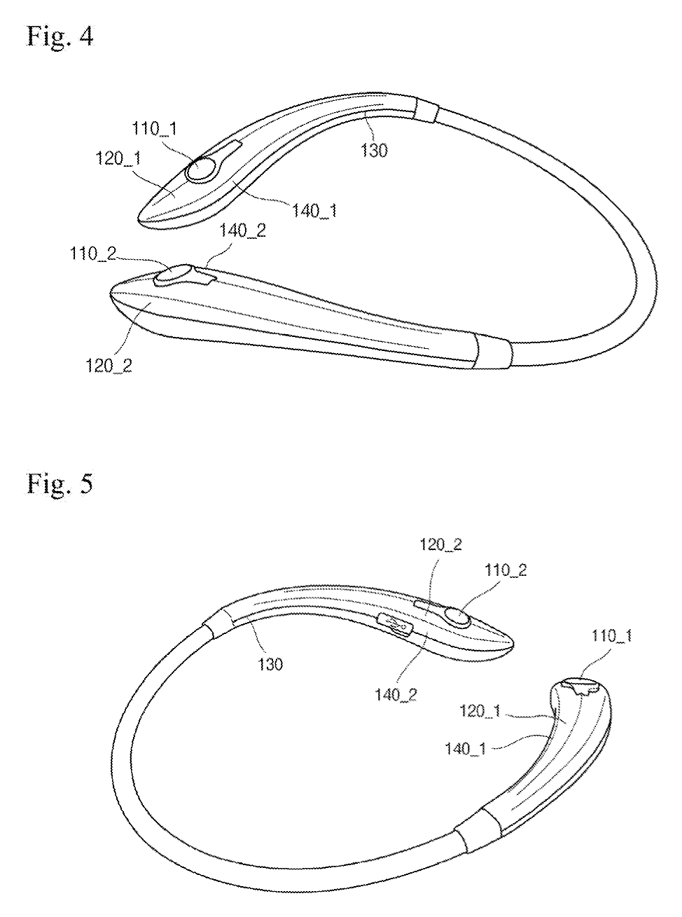

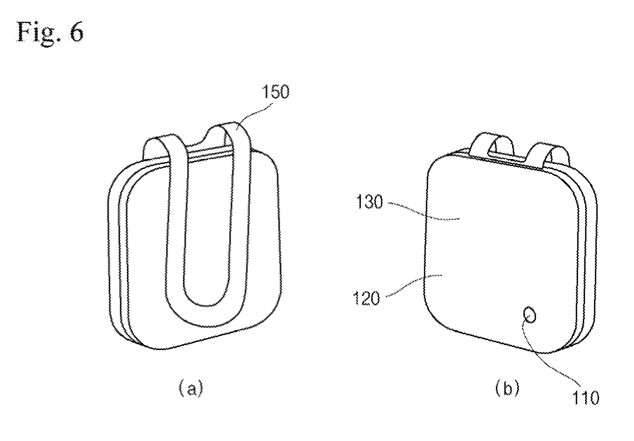

The sound alerting apparatus 100 may sense an ambient sound, analyze the sound, and operate according to the sensitivity of the sound and operation setting information received from the terminal 200. The sound alerting apparatus 100 may be configured as a neckband-type headphone as shown in FIGS. 3 to 5 or a clip as shown in FIG. 6.

An example in which the sound alerting apparatus 100 is implemented and operated as a neckband-type headphone is described below in detail with reference to FIGS. 3 to 5, and an example in which the sound alerting apparatus 100 is implemented and operated as a clip is described below in detail with reference to FIG. 6.

The sound alerting apparatus 100 may sense an ambient sound, compare the sensitivity of the sound with a vibration reference sensitivity included in the operation setting information, and operates a vibration generator according to a result of the comparison.

According to an embodiment of the present invention, when the sensitivity of the ambient sound is the reference sensitivity or higher, the sound alerting apparatus 100 operates the vibration generator to alert the user to a sound occurrence.

There may be provided a plurality of vibration generators, e.g., two vibration generators.

The sound alerting apparatus 100 may calculate a gap in sensitivity between sounds respectively received from sound sensors and may operate all or some of the plurality of vibration generators depending on whether the sensitivity gap between the received sound is a predetermined value or higher, alerting the user to a sound occurrence.

When the sensitivity gap between the sounds respectively received from the sound sensors is the predetermined value or higher, the sound alerting apparatus 100 may identify the direction of a sound sensor from which a higher-sensitivity sound is received among the sound sensors and operate the vibration generator corresponding to the direction, alerting the user to a sound occurrence in the direction.

The sound alerting apparatus 100 generates a vibration according to a vibration time and strength previously set by the user when operating the vibration generator.

For example, when the sensitivity of an ambient sound received, e.g., a honk, doorbell sound, or baby cry, is higher than a sound sensitivity previously set by the user, the user may be alerted to the direction. This may benefit the user, especially when the user suffers from a hearing loss or impairment.

When the sensitivity gap between the sounds received from the sound sensors is the predetermined value or lower, the sound alerting apparatus 100 may determine that the sounds respectively received from the sound sensors have a similar sensitivity and operate all of the vibration generators, alerting the user to a sound occurrence.

The terminal 200 may be a terminal carried by the user using the sound alerting apparatus 100, which may be implemented as a personal digital assistant (PDA), cellular or mobile phone, or smartphone, or any other various portable devices.

The terminal 200 may have an application previously installed thereon to set or control operations of the sound alerting apparatus 100. Alternatively, the terminal 200 may download the application through a predetermined route, e.g., from a server.

The application may automatically run when the sound alerting apparatus 100 is connected with the terminal 200 via, e.g., wireless communication (e.g., Bluetooth, ZigBee, or Wi-Fi, or infrared (IR)) or wired communication.

The application may provide a process for configuring the operation setting information of the sound alerting apparatus 100, enabling the user to set an operation of the sound alerting apparatus 100. The operation setting information of the sound alerting apparatus 100 may include a vibration reference sensitivity, a vibration time, a sound sensitivity gap, and a vibration strength.

The vibration reference sensitivity, as used herein, may mean a sensitivity serving as a reference for generating a vibration depending on the sensitivity of an ambient sound recognized.

The vibration reference sensitivity may be set by the user or may be automatically on the application. When set automatically on the application, the vibration reference sensitivity may be determined in reference to a per-sound sensitivity context information table. The per-sound sensitivity context information table may be a table retaining context information per sound sensitivity.

For example, the per-sound sensitivity context information table may store information indicating that, when the sensitivity of a baby cry is level 1, the baby babbles, information indicating that, when the sensitivity of a baby cry is level 3, the bay is hungry and asks to change diapers, and information indicating that, when the sensitivity of the baby cry is level 5, the baby is under emergency.

The vibration reference sensitivity may be recommended on the application. For example, the terminal 200 may receive the type of a sound required to be alerted from the user, extract a sound required by the user in reference to the per-sound sensitivity context information table, set the vibration reference sensitivity to a mean value of sensitivities corresponding to context information required to be alerted using the per-sound sensitivity context information, and recommend the vibration reference sensitivity for the user.

The vibration time, as used herein, may mean a time when the sound alerting apparatus 100 is required to generate a vibration when the sensitivity of an ambient sound received is the vibration reference sensitivity or higher.

The sound sensitivity gap, as used herein, may mean a gap or difference in sensitivity between sounds respectively received from or through the sound sensors disposed in the sound alerting apparatus 100. The reason for setting the sound sensitivity gap is to operate all or only some of the vibration generators disposed in the sound alerting apparatus 100 by determining whether the sound sensitivity gap is a predetermined value or higher or lower.

When the sound sensitivity gap is a predetermined value or higher, the vibration generator positioned in the direction where a higher sound sensitivity is measured among the vibration generators in the sound alerting apparatus 100 is operated to alert the user to a sound occurrence in the direction. When the sound sensitivity gap is the predetermined value or lower, all of the vibration generators in the sound alerting apparatus 100 are operated to alert the user to a sound occurrence.

The vibration strength, as used herein, may mean the strength of a vibration generated by the sound alerting apparatus 100 when the sensitivity of an ambient sound received is the vibration reference sensitivity or higher.

FIG. 2 is a block diagram illustrating a sound alerting apparatus according to an embodiment of the present invention.

Referring to FIG. 2, the sound alerting apparatus 100 includes a microphone 110, a vibration generator 120, and a controller 130.

The microphone 110 may receive and signal-process an ambient sound and provides the signal-processed sound to the controller 130.

Although the microphone 110 and the vibration generator 120 each are shown in a single block, there may be provided a plurality of microphones 110 (e.g., two microphones 110) and a plurality of vibration generators 120 (e.g., two vibration generators 120).

Each of the vibration generators 120 generates a vibration of a predetermined strength for a predetermined time under the control of the controller 130.

The vibration generator 120 may be disposed on the sound alerting apparatus 100, corresponding to the direction of the microphone 110.

The controller 130 may measure the sensitivity of a sound received through the microphone 110, compare the sensitivity of the sound with a preset vibration reference sensitivity, and operate the vibration generator 120 depending on a result of the comparison.

According to an embodiment of the present invention, when the sensitivity of the sound is the vibration reference sensitivity or higher, the controller 130 may operate to alert the user to a sound occurrence.

The controller 130 may calculate a gap in sensitivity between the sounds respectively received from the microphones 110 and operate all or some of the vibration generators 120 depending on whether the sound sensitivity gap is a predetermined value or higher, alerting the user to a sound occurrence.

When the gap in sensitivity between the sounds respectively received from the microphones 110 is the predetermined value or higher, the controller 130 may identify the direction of the microphone 110 through which a higher sensitivity of sound is received among the microphones 110 and operate the vibration 120 corresponding to the direction to alert the user to a sound occurrence in the direction.

The controller 130 may operate each vibration generator 120 to generate a vibration depending on a vibration time and strength previously set by the user.

For example, when the sensitivity of an ambient sound received, e.g., a honk, doorbell sound, or baby cry, is higher than a sound sensitivity previously set by the user, the direction may be known to the user through the vibration, helping the user, e.g., when the user suffers from a hearing loss.

FIGS. 3 to 5 are views illustrating a sound alerting apparatus implemented as a neckband according to an embodiment of the present invention.

Referring to FIGS. 3 to 5, the sound alerting apparatus 100 may be configured or implemented to be wearable on the user's neck.

Referring to FIG. 3, the sound alerting apparatus 100 includes a first microphone 110_1 and a second microphone 110_2 respectively positioned at both sides of the user's neck, a first vibration generator 120_1 disposed corresponding to a direction of the first microphone 110_1 and a second vibration generator 120_2 disposed corresponding to a direction of the second microphone 110_2, and a controller 130. The sound alerting apparatus 100 may further include a first hearing-aid earphone 140_1 and a second hearing-aid ear phone 140_2.

The first microphone 110_1 and the second microphone 110_2 each may receive an ambient sound, signal-process the ambient sound, and provide the signal-processed sound to the controller 130.

The first vibration generator 120_1 and the second vibration generator 120_2 may be simultaneously operated under the control of the controller 130 to generate vibrations, or only one of the first vibration generator 120_1 and the second vibration generator 120_2 may be generated under the control of the controller 130 to generate a vibration.

The controller 130 may measure the sensitivity of sounds received through the first microphone 110_1 and the second microphone 110_2, compare the sound sensitivity with a preset vibration reference sensitivity, and operate the first vibration generator 120_1 and the second vibration generator 120_2 according to a result of the comparison.

According to an embodiment of the present invention, when the sound sensitivity is the vibration reference sensitivity or higher, the controller 130 may operate at least one of the first vibration generator 120_1 and the second vibration generator 120_2 to alert the user to the occurrence of the sounds.

The controller 130 may calculate a gap in sensitivity between sounds respectively received from the first microphone 110_1 and the second microphone 110_2 and may operate all or one of the first vibration generator 120_1 and the second vibration generator 120_2 depending on whether the sound sensitivity gap is a predetermined value or higher, alerting the user to a sound occurrence.

When the gap in sensitivity between the sounds respectively received from the first microphone 110_1 and the second microphone 110_2 is the predetermined value or higher, the controller 130 may identify the direction of the microphone from which a higher sensitivity of sound is received of the first microphone 110_1 and the second microphone 110_2 and operates the vibration generator corresponding to the direction, alerting the user to a sound occurrence in the direction.

For example, when a higher sensitivity of sound is received through the first microphone 110_1 than the second microphone 110_2, the controller 130 may operate the first vibration generator 120_1 disposed corresponding to the direction of the first microphone 110_1, alerting the user to a sound occurrence in the direction.

As another example, when a higher sensitivity of sound is received through the second microphone 110_2 than the first microphone 110_1, the controller 130 operates the second vibration generator 120_2 disposed corresponding to the direction of the second microphone 110_2, alerting the user to a sound occurrence in the direction.

The first hearing-aid earphone 140_1 and the second hearing-aid earphone 140_2, respectively, output sounds received from the first microphone 110_1 and the second microphone 110_2. The first hearing-aid earphone 140_1 and the second hearing-aid earphone 140_2 each may function as hearing aids or provide a hearing-aid function, as does a general hearing-aid device, thus eliminating the need for the user to keep wearing the earphone.

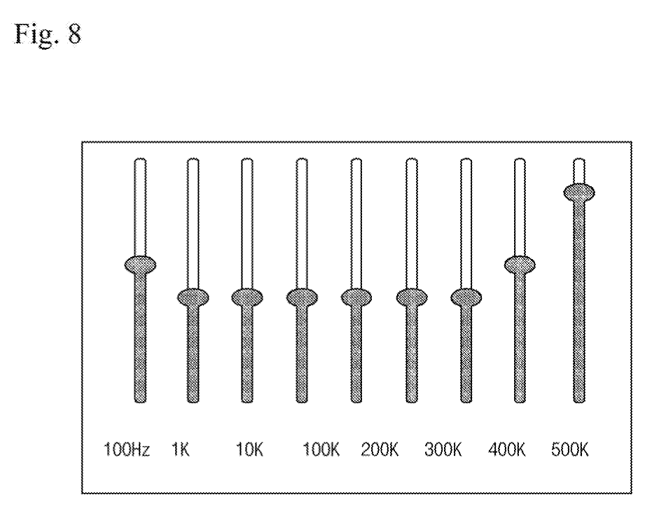

The first hearing-aid earphone 140_1 and the second hearing-aid earphone 140_2 each may have a voice band spectrum set by the user through a process described below in connection with FIG. 8, and each may operate according to the voice band spectrum to provide the user with the functionality of a common hearing-aid device.

FIG. 6 is a view illustrating a sound alerting apparatus implemented as a clip according to an embodiment of the present invention.

Referring to FIG. 6, the sound alerting apparatus 100 may be implemented in a clip shape to be wearable on a particular portion of the user.

Referring to (a) of FIG. 6, the sound alerting apparatus 100 implemented as a clip may be attached or fastened to a portion of the user's clothes. Referring to (b) of FIG. 6, the sound alerting apparatus 100 includes a microphone 110, a vibration generator 120, and a controller 130.

The microphone 110 receives an ambient sound, signal-processes the sound, and provides the signal-processed sound to the controller 130.

There may be provided a plurality of vibration generators 120, e.g., two vibration generators.

The plurality of vibration generators 120 may be simultaneously operated under the control of the controller 130 to generate vibrations.

The controller 130 may measure the sensitivity of the sound received through the microphone 110, compare the sound sensitivity with a preset vibration reference sensitivity, and operate the vibration generator 120 according to a result of the comparison.

According to an embodiment of the present invention, when the sensitivity of an ambient sound received is a sound sensitivity set by the user or higher, a direction may be known to the user through a vibration, helping the user who is. e.g., a hearing-impaired person.

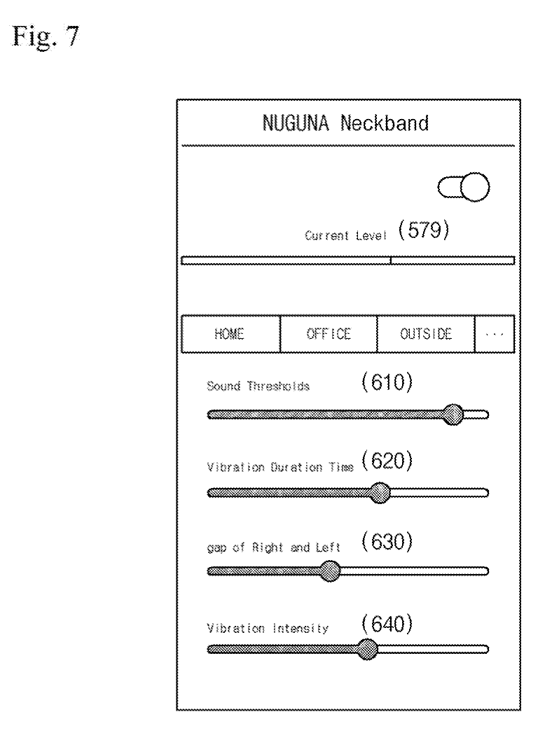

FIGS. 7 and 8 are views illustrating screenshots of an application running on a terminal according to an embodiment of the present invention.

Referring to FIGS. 7 and 8, the terminal 200 may have an application previously installed thereon, enabling settings of operations of the sound alerting apparatus 100. Alternatively, the application may be downloaded through a predetermined route (e.g., from a server) and installed on the terminal 200.

The application may be automatically run when the sound alerting apparatus 100 is connected with the terminal 200 via wireless communication (e.g., Bluetooth, ZigBee, Wi-Fi, or IR transmission) or wired communication.

The application may provide a process for configuring operation setting information of the sound alerting apparatus 100, allowing the user to set operations of the sound alerting apparatus 100. The operation setting information of the sound alerting apparatus 100 may include a vibration reference sensitivity 610, a vibration time 620, a sound sensitivity gap 630, and a vibration strength 640.

The vibration reference sensitivity 610 may mean a sensitivity serving as a reference for generating a vibration depending on the sensitivity of an ambient sound recognized. The vibration reference sensitivity 610 may be set by the user or may be automatically on the application. When set automatically on the application, the vibration reference sensitivity 610 may be determined in reference to a per-sound sensitivity context information table. The per-sound sensitivity context information table may be a table retaining context information per sound sensitivity.

For example, the per-sound sensitivity context information table may store information indicating that, when the sensitivity of a baby cry is level 1, the baby babbles, information indicating that, when the sensitivity of a baby cry is level 3, the bay is hungry and asks to change diapers, and information indicating that, when the sensitivity of the baby cry is level 5, the baby is under emergency.

The vibration reference sensitivity 610 may be recommended on the application. For example, the terminal 200 may receive the type of a sound required to be alerted from the user, extract a sound required by the user in reference to the per-sound sensitivity context information table, set the vibration reference sensitivity 610 to a mean value of sensitivities corresponding to context information required to be alerted using the per-sound sensitivity context information, and recommend the vibration reference sensitivity for the user.

The vibration time 620 may mean a time when the sound alerting apparatus 100 is required to generate a vibration when the sensitivity of an ambient sound received is the vibration reference sensitivity 610 or higher.

The sound sensitivity gap 630 may mean a difference in sensitivity between sounds respectively received through the sound sensors disposed in the sound alerting apparatus 100. The reason for setting the sound sensitivity gap 630 is to operate all or only some of the vibration generators disposed in the sound alerting apparatus 100 by determining whether the sound sensitivity gap 630 is a predetermined value or higher or lower.

The vibration strength 640 may mean the strength of a vibration generated by the sound alerting apparatus 100 when the sensitivity of an ambient sound received is the vibration reference sensitivity 610 or higher.

The application running on the terminal 200 may be configured to set a hearing-aid earphone (e.g., the first hearing-aid earphone 140_1 or second hearing-aid earphone 140_2 shown in FIGS. 3 to 6) as illustrated in FIG. 8, allowing for settings of a voice band spectrum of a sound at which the user who is a hearing-impaired person is difficult to hear so that the hearing-aid earphone is operated according to the set voice band spectrum to function as a hearing aid.

Although the present invention has been shown and described in connection with specific embodiments, it should be noted that various changes may be made thereto without departing from the scope of the invention. Hence, the scope of the invention should not be limited to the described embodiments, but rather be defined by the appended claims and equivalents thereof.

What is claimed is:

1. A method for providing a notification service for hearing-impaired persons, executed on a sound alerting apparatus, the method comprising:
determining whether sensitivities of ambient sounds respectively received through sound sensors are a preset vibration reference sensitivity or higher;
calculating a gap between the sensitivities when the sensitivities are the preset vibration reference sensitivity or higher, and
operate at least one of vibration generators according to the gap to alert a user to a sound occurrence.

2. The method of claim 1, wherein calculating the gap includes, when the gap is a predetermined value or less, operating the vibration generators to alert the user to the sound occurrence, and when the gap is higher than the predetermined value, identifying a direction of one receiving a higher sensitivity of sound of the sound sensors.

3. The method of claim 2, wherein identifying the direction of one receiving the higher sensitivity of sound of the sound sensors includes operating one of the vibration generators which is disposed corresponding to the sound sensor receiving the higher sensitivity of sound to alert the user to the sound occurrence in the identified direction.

4. The method of claim 1, wherein operating the at least one of vibration generators according to the gap to alert the user to the sound occurrence includes operating at least one of the vibration generators according to a preset vibration strength and time to alert the user to the sound occurrence.

5. The method of claim 1, further comprising outputting each of the ambient sounds through an earphone.

6. A sound alerting apparatus, comprising:
microphones receiving and processing ambient sounds;
vibration generators each operated under a control signal to generate a vibration; and
a controller determining whether sensitivities of the ambient sounds respectively received from the microphones are a preset vibration reference sensitivity or higher, calculating a gap between the sensitivities when the sensitivities are the preset vibration reference sensitivity or higher, and operating at least one of the vibration generators according to the gap to alert a user to a sound occurrence.

7. The sound alerting apparatus of claim 6, wherein when the gap is a predetermined value or less, the controller operates the vibration generators to alert the user to the sound occurrence, and when the gap is higher than the predetermined value, the controller identifies a direction of one receiving a higher sensitivity of sound of the sound sensors.

8. The sound alerting apparatus of claim 7, wherein the controller operates one of the vibration generators which is disposed corresponding to the sound sensor receiving the higher sensitivity of sound to alert the user to the sound occurrence in the identified direction.

9. The sound alerting apparatus of claim 6, wherein the controller operates at least one of the vibration generators according to a preset vibration strength and time to alert the user to the sound occurrence.

10. The sound alerting apparatus of claim 6, further comprising an earphone outputting each of the ambient sounds to the user.
